(19) United States
(12) Patent Application Publication
(10) Pub. No.: US 2011/0230857 A1
(43) Pub. Date: Sep. 22, 2011

(54) ELECTRONIC DELIVERY SYSTEMS AND METHODS WITH FEEDBACK

(76) Inventor: Ewa Herbst, Edgewater, NJ (US)

(21) Appl. No.: 12/098,257

(22) Filed: Apr. 4, 2008

Related U.S. Application Data
(63) Continuation-in-part of application No. 10/608,829, filed on Jun. 26, 2003, now Pat. No. 7,526,334.
Application No. 10/608,829 is a continuation of application No. 09/733,023, filed on Dec. 8, 2000, now Pat. No. 6,708,066.
Application No. 09/733,023 is a continuation-in-part of application No. 11/151,967, filed on Jun. 14, 2005, now abandoned.
Application No. 11/151,967 is a continuation-in-part of application No. 11/213,050, filed on Aug. 26, 2005.
Application No. 11/213,050 is a continuation of application No. 10/706,844, filed on Nov. 12, 2003, now abandoned.
Application No. 10/706,844 is a continuation of application No. 09/507,873, filed on Feb. 22, 2000, now Pat. No. 6,684,106.
Application No. 09/507,873 is a continuation of application No. 09/013,049, filed on Jan. 27, 1998, now Pat. No. 6,029,090.
(60) Provisional application No. 60/599,959, filed on Aug. 9, 2004.
(60) Provisional application No. 60/034,869, filed on Jan. 27, 1997.
(60) Provisional application No. 60/915,097, filed on Apr. 30, 2007.

(51) Int. Cl. A61M 31/00; A61N 1/36

(52) U.S. Cl. 604/503; 604/66; 607/43; 607/46; 607/62; 607/50; 607/39

(57) ABSTRACT

In accordance with some embodiments of the present invention, delivery systems and methods for delivering a substance in a medium is provided. The delivery system may include a delivery component, an electronic controller, one or more sensors, and a feedback mechanism. The delivery component has at least one reservoir that contains a substance (e.g., a liquid, a drug, etc.). The electronic controller is connected to the delivery component and controls the release of the substance from the at least one reservoir into the medium. The sensors monitor one or more parameters in the medium and provide a signal to a feedback mechanism. The feedback mechanism, in response to the signal, sends an electrical signal that directs the controller to release the substance from the reservoir into the medium, thereby maintaining a desire level of a monitored parameter (e.g., pH, pO₂, temperature, pressure, ions, volume, impedance, dosage, biomarkers, etc.).

### CROSS-REFERENCE TO RELATED APPLICATIONS

This application claims the benefit of and priority under 35 U.S.C. §119(e) to U.S. Provisional Patent Application No. 60/915,097, filed Apr. 30, 2007, which is hereby incorporated by reference herein in its entirety. This application claims priority, under 35 U.S.C. §120, to, and is a continuation-in-part of, U.S. patent application Ser. No. 11/151,967, filed Jun. 14, 2005, which claimed priority under 35 U.S.C. §119(e) to U.S. Provisional Patent Application No. 60/599,959, filed Aug. 9, 2004, both of which are hereby incorporated by reference herein in their entirety. This application is also a continuation-in-part of U.S. patent application Ser. No. 11/213,050, filed on Aug. 26, 2005, and to which this application claims priority under 35 U.S.C. §120. U.S. patent application Ser. No. 11/213,050 is a continuation of U.S. patent application Ser. No. 10/706,844, filed on Nov. 12, 2003, which is a continuation of U.S. patent application Ser. No. 09/507,873, filed on Feb. 22, 2000, now U.S. Pat. No. 6,684,106, which is a continuation of U.S. patent application Ser. No. 09/013,049, filed Jan. 27, 1998, now U.S. Pat. No. 6,019,090, which claimed priority under 35 U.S.C. §119(e) to U.S. Provisional Patent Application No. 60/034,869, filed on Jan. 27, 1997, all of which are incorporated by reference herein in their entireties. This application is also a continuation-in-part of U.S. patent application Ser. No. 10/608,829, filed on Jun. 26, 2003, and to which this application claims priority under 35 U.S.C. §120. U.S. patent application Ser. No. 10/608,829 is a continuation of U.S. patent application Ser. No. 09/733,023, filed Dec. 8, 2000, now U.S. Pat. No. 6,708,066, which claimed priority to PCT Application No. US99/29564, filed Dec. 10, 1999 and designates the United States of America among other countries, all of which are incorporated by reference herein in their entireties.

### BACKGROUND OF INVENTION

1. Field of Invention

This invention relates generally to delivery systems that transport substances (e.g., drugs, fluids, etc.), where the delivery may be adjusted and controlled using a feedback mechanism.

2. Status of Prior Art

Electrical stimulation (ES) is widely used in biological and bio-medical research as well as in diagnostics and in clinical treatment. In faradic stimulation an intermittent or a continuous direct or alternating current or voltage is produced, whereas in electromagnetic stimulation, a current passing through a coil produces an electromagnetic field whose pattern depends on the waveform of the current.

Electrical stimulation is employed, in neuromuscular research and other suitable medical applications, in medical diagnosis and treatment (e.g., of neurological or neuropsychiatric diseases such as addiction, anxiety, and cognitive, eating, mood, and sleep disorders), and in laboratory equipment, such as, for pulsed voltage electrophoresis and other suitable applications. Such stimulation is also used in connection with bone healing and for wound healing, as well as for pain relief by means of transcutaneous electrical nerve stimulation (TENS) as well as spinal cord stimulation. The use of ES to effect nerve regeneration is disclosed in the art, for example in one or more of the patents or patent applications referred to above.

Researchers in the biological and medical sciences, physiotherapists, and clinicians who make use of ES require electrical stimulators of a type suitable for the activities in which they are engaged. Thus neurological investigators who seek to non-invasively stimulate deep nerves, e.g. to correct motor disorders related to Parkinson's disease, make use of commercially available magnetic stimulators which produce a high-intensity magnetic field pulse for this purpose.

Also commercially available are constant current stimulators for direct cortical stimulation as well as electrical stimulators for nerve (e.g., cavernous, gastric, occipital, phrenic, sacral, vagus, etc.) and muscle stimulation procedures which generate e.g., single or double pulses, or trains of such pulses. And commercially available are wave generators capable of selectively generating e.g., sine and square wave pulses suitable for other types of electrical stimulation.

Electrical stimulator components, multi-functional systems, and methods for generating output signals are described, for example, in above-mentioned U.S. Pat. No. 6,684,106, which is hereby incorporated by reference herein in its entirety.

Accordingly, it is desirable to use these electrical stimulator systems or components to improve delivery systems, including drug delivery, by providing the capability to electrically control the delivery of substances.

### SUMMARY OF INVENTION

In accordance with some embodiments of the present invention, delivery systems and methods for delivering a substance in a medium, e.g., blood, saliva, other body fluids, muscle, etc., are provided.

In some embodiments, the delivery system includes a delivery component that includes at least one reservoir. The reservoir contains a substance (e.g., a liquid, a drug, etc.). The delivery system also includes an electronic controller that is connected to the delivery component and that controls the release of the substance from the at least one reservoir into the medium. The delivery system also includes a sensing mechanism (e.g., one or more sensors, one or more biomarkers, etc.) that monitors one or more parameters in the medium and provides a signal to a feedback mechanism. The feedback mechanism, in response to the signal, sends an electrical signal that directs the controller to effect the release of the substance from the reservoir into the medium.

In another embodiment of the present invention, the delivery system may include a delivery component that has a plurality of reservoirs. A controller that is connected to the delivery component that includes a signal generator, a selection mechanism, and a plurality of output terminals. The signal generator generates a plurality of signals. Each signal has a controllable waveform based on one or more electrical parameters (e.g., amplitude, frequency, shape, timing parameters, phase, pulse duration, and pulse repetition rate). The selection mechanism selects as output signals one or more of the plurality of signals generated by the signal generator. The controller is also configured to place at least one of the output signal on each of the output terminals. It should be noted that the output signals control the release of the substance from the plurality of reservoirs. It should also be noted that each reservoir may contain a different substance. The delivery system may also include at least one feedback mechanism that directs the controller to effect the release of at least one of the substances from the plurality of reservoirs into the medium.

Because the delivery system may be controlled by the controller and the feedback mechanism, the delivery system is capable of precision mixing of various fluids or drugs (e.g., flow control of substances contained in multiple reservoirs), time delivery synchronization (e.g., daily doses, intermittent, maintenance of treatment on a daily basis, or administer the substance when needed, etc.), volumetric control of substances, synchronization for multiple substances, diluting substances for providing the appropriate dose, controlling different ports, etc.

There has thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF DRAWINGS

Various objects, features, and advantages of the present invention can be more fully appreciated with reference to the following detailed description of the invention when considered in connection with the following drawings, in which like reference numerals identify like elements.

FIG. 1 shows a schematic diagram of an illustrative system suitable for delivering a substance (e.g., drugs, liquid, etc.) and having feedback capabilities in accordance with some embodiments of the present invention.

FIG. 2 is an illustrative block diagram of an exemplary electrical stimulation system having an ES stage, an output stage, and a measuring stage that may be used in accordance with some embodiments of the present invention.

FIG. 3 is an illustrative block diagram of a basic version of a system for producing various electrical stimulation pulses that may be used in accordance with some embodiments of the present invention.

FIG. 4 is an illustrative example of a preset custom module that may be used in accordance with some embodiments of the present invention.

### DETAILED DESCRIPTION OF INVENTION

In the following description, numerous specific details are set forth regarding the methods and systems of the present invention and the environment in which such methods and systems may operate, etc., in order to provide a thorough understanding of the present invention. It will be apparent to one skilled in the art, however, that the present invention may be practiced without such specific details, and that certain features which are well known in the art are not described in detail in order to avoid complication of the subject matter of the present invention. In addition, it will be understood that the examples provided below are exemplary, and that it is contemplated that there are other methods and systems that are within the scope of the present invention.

Generally speaking, the present invention is directed to methods and systems for delivering substances (e.g., drugs, fluids, etc.), where the delivery may be automatically controlled using an electrical stimulator component and/or a feedback mechanism.

The delivery system generally includes a waveform or pulse generator circuit (e.g., a digital pulse generator, an electrical stimulator component, etc.), one or more sensors, and at least one feedback mechanism (e.g., a closed feedback loop, an open feedback loop, etc.). The sensors (e.g., physical sensors, chemical sensors, biochemical sensors, etc.) detect and/or monitor various conditions in a medium (e.g., prevailing in the tissue, existing in a system, etc.) and provide the detected information to the feedback mechanism. In turn, the feedback mechanism may direct the controller to perform an operation in response to the detected information (e.g., open a reservoir or release a given amount of drugs using an electrical, mechanical, optical, or other signal). For example, the electrical stimulator component may adjust the signal generated by the electrical stimulator component to maintain an optimal operating or treatment condition. In particular, the electrical stimulator component may adjust the signal generated to maintain a given level of, e.g., ion, salt, pH, pO₂, biochemical marker, volume, dosage, etc. or a gradient thereof When used for treatment or other suitable biomedical applications, such an adjustment may optimize the outcome of the treatment.

As used herein, “biochemical marker,” which may be used interchangeably with “biological marker” or “biomarker,” refers to a characteristic that is measured and evaluated as an indicator of a biological process (e.g., a normal biological process, a disease or abnormal condition, or a response to a pharmacologic or other therapeutic treatment or procedure for drug discovery). Such biomarkers may include, but are not limited to, single nucleotide polymorphisms, gene expression patterns, proteins, peptides, and small molecule metabolites (e.g., glucose, cholesterol, phospholipids, amino acids, vitamins, etc.).

The delivery system of the present invention may be used for a number of clinical applications, such as, for example, peripheral nerve regeneration, spinal cord regeneration, bone healing, wound healing, cancer treatment, cardiac rhythm management, an artificial pancreas, drug delivery, stem cell delivery, or pre-treated stem cell delivery (e.g., using the electrical stimulation systems to effect the stem cells or gene therapy), in addition to the neurological and functional electrical stimulation applications.

In some embodiments, the delivery system may be used for bone healing. Electrical stimulation of bone healing has been used clinically for more than about three decades. One approach for bone healing may include using implantable devices for both long bone healing and for spinal fusion. By controlling the electrical potential of the stimulating electrode (cathode) to be below the hydrogen evolution level, the optimization of the bone formation process may be achieved. This may be accomplished by measuring pH in the immediate vicinity of the cathode (which indicates if the hydrogen evolution takes place) and adjusting the electrical potential of the stimulating electrode accordingly.

In another suitable embodiment, the delivery system may be used for the electrochemical treatment of cancer. Prior methods that were developed originally in Sweden and used in treatment of thousands of patients in China deliver electric current to solid superficial tumors via several indwelled electrodes. The generated current destroys the tumor without any extensive bleeding by causing necrosis of the tissue and significantly changed pH values in the tissue. However, an improved method for the electrochemical treatment of cancer may be provided by automatically adjusting the electrode potential based at least in part on a measured pH value in the tissue to result in a smaller but sufficient pH changes to affect the tumor, thereby minimizing serious necrotic and inflammatory reactions.

It should be noted that, in some embodiments, electrical stimulation may be provided on demand. Electrical stimulation may be used for tissue regeneration, for recovery or maintenance of function (e.g., upper and lower extremity function, bladder, bowel, and erectile function, and respiratory function, which may be impaired in connection with, e.g., spinal cord injury, stroke, head injury, cerebral palsy, or multiple sclerosis), as well as for cardiac rhythm management. In all of those cases some disease-specific or therapeutic response-specific physical, chemical, or biochemical markers may be measured, resulting in on-demand delivery of a specific sequence of an electric signal from an external or implantable device. In the case of implantable devices, electrical stimulation may be administered as needed over a long period of time, initially to promote tissue regeneration or return of function and, at a later time, to maintain it.

In yet another suitable embodiment, the delivery system may be used to provide controlled, active drug delivery. External or implantable drug delivery systems may be automatically controlled by electrical signals received in response to appropriate physical, chemical, or biochemical markers continuously or intermittently monitored in the tissue. A waveform generator may be used to control both the drug delivery timing sequences and a sensor measurement sequence, if intermittent. Sensor information may trigger the drug delivery mechanism, when, for example, the measured parameters fall outside of a given optimal value range (e.g., a preset optimal value range). For example, an example of a specific application may be an insulin pump.

Alternatively, the delivery system of the present invention may be used to deliver fluids or other suitable substances (e.g., through catheters, IV, or microfluidic channels). For example, the delivery system may be fabricated or integrated onto a System-on-a-Chip (SoC). In addition, sensors, an electrical stimulator, a microprocessor (if needed) for controlling various components on the SoC, and/or any other suitable component may be integrated onto the delivery system, which may be implemented as a system on the chip.

For example, microfluidic devices may be used in printheads and other components of an inkjet printer. The present invention may be used to control the delivery of ink at the micron scale. As described above, each device may be electrically controlled using the delivery system of the present invention. In response to receiving an electric or optical signal a reservoir may open, allowing ink to flow through one of the channels in the inkjet printhead. The system can be also used for delivering antibodies, etc. for “diagnostic arrays” or “lab on a chip”.

In some embodiments, the delivery system of the present invention may be used in a high pressure liquid chromatography (HPLC) system. While HPLC has provided a separation tool capable of high resolution separations, rapid analysis, and high sensitivity, obtaining all the benefits that HPLC offers typically requires careful attention to the flow portion of the HPLC system that carries the sample. Limitations on the performance of the HPLC system are generally caused by problems with fluid transfer (e.g., dead volume, volume is different in certain columns, etc.). In many cases, the smallest amount of dead volume in the HPLC system may make the difference between an acceptable separation and an unacceptable one. Dead volume dilutes chromatographic peaks, increasing their volume and reducing chromatographic efficiency. Accordingly, the delivery system of the present invention may be used in a HPLC system to control the flow of fluids throughout the system, thereby reducing the amount of dead volume in the HPLC system.

In some embodiments, the delivery system of the present invention may be used in a bioreactor. A bioreactor is generally a vessel or contained area in which a chemical process that involves organisms or biochemically active substances derived from such organisms is carried out. In particular, the environmental conditions within the vessel of the bioreactor, such as gas (e.g., air, oxygen, nitrogen, carbon dioxide), flow rates, temperature, pH, dissolved oxygen levels, and agitation speed need to be closely monitored and controlled. The delivery system may be integrated or connected to the bioreactor to monitor and control the environmental conditions in the bioreactor.

It should also be noted that although the present invention is described in terms of a delivery system, this is only one embodiment. The present invention may also be used in analytical instrumentation, in automatic test equipment, for drug discovery, or any other suitable application.

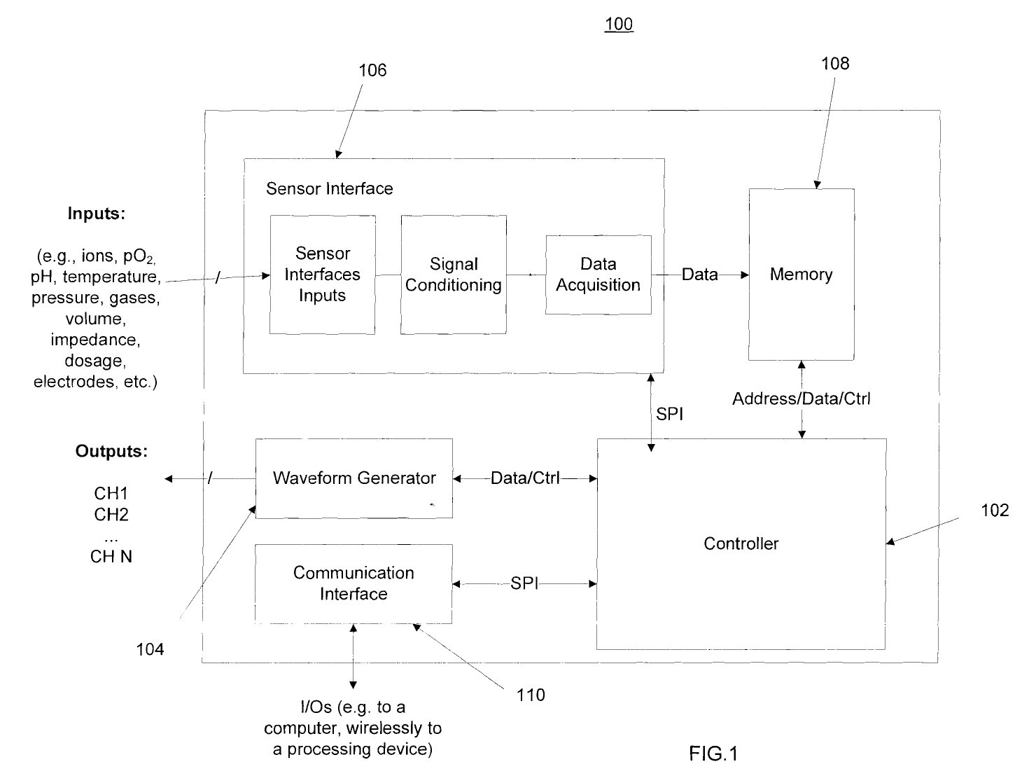

FIG. 1 shows a schematic diagram of an illustrative system suitable for delivering a substance (e.g., drugs, liquid, etc.) and having feedback capabilities in accordance with some embodiments of the present invention. As shown in FIG. 1, delivery system 100 includes a controller 102 (e.g., a microprocessor or any other suitable processing device), a waveform generator 104, and a sensor interface 106.

In some embodiments, delivery system 100 may also include memory 108 and a communication interface 110. Communication interface 110 may be any communication link suitable for communicating data between delivery system 100 and a computer (e.g., a workstation, PDA, laptop computer, cellular telephone, etc.), such as a network link, a dial-up link, a wireless link, a hard-wired link, etc.

In some embodiments, waveform generator 104 may be a digital pulse generator Application Specific Integrated Circuit (ASIC). In some embodiments, waveform generator 104 may be an electrical stimulator component (sometimes referred to herein as an “ES component”) with one or more output stages.

In general, an electrical stimulator component includes an ES signal stage having a selector coupled to a plurality of different signal generators, each producing a signal having a distinct shape such as a sine, a square or sawtooth wave, or a simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. In some embodiments, also included in the system may be a measuring stage which measures and displays (if needed) the electrical stimulation signal operating on the substance being treated as well as the outputs of various sensors which sense conditions prevailing in this substance, whereby the user of the system can manually adjust it or have it automatically adjusted by feedback to provide an electrical stimulation signal of whatever type he wishes and the user can then observe the effect of this signal on a substance being treated.

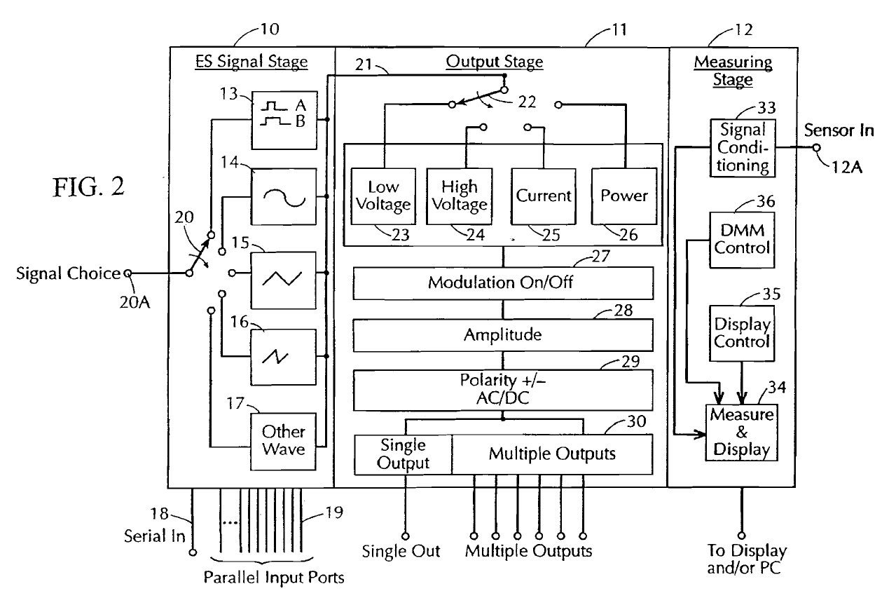

An electrical stimulator component or system in accordance with the present invention in shown in FIG. 2. The electrical stimulator system is composed of an ES signal stage 10 which at the user's discretion generates a faradic, an electromagnetic, or other type of electrical stimulation signal which is fed to an output stage 11. Output stage 11 processes the electrical stimulation signals selected by the user to yield a stimulation signal suitable for its intended biological or biomedical application.

Also provided is a measuring stage 12 which measures and displays the electrical stimulation signal operating on the biological substance being subjected thereto, and/or its electrical parameters as well as the output of various sensors which sense conditions prevailing in this substance whereby the user is able to observe, monitor as well as to adjust the effects of the stimulation signal he has selected on the substance being treated.

ES signal stage 10 includes signal generators 13 to 17 producing signals of different shape. Generator 13 is a pulse wave generator generating one or more rectangular pulses, such as pulses A and B of different width which can be outputted separately or can be added or subtracted from each other to yield A or B, A plus B or A minus B. Generator 14 is a sine wave generator, generator 15 generates a triangular or sawtooth wave, and generator 16 produces a ramp voltage wave. Generator 17 yields a wave of any arbitrary shape. The signal generators are capable of generating a minimum one pulsatory signal or a greater numbers of pulsatory signals, or of generating a gated signal with a minimum of one period or a greater number of periods, with individual adjustments of electrical parameters.

By means of a serial input port 18 to ES stage 10 or a set of parallel input ports 19, the parameters of the respective waves produced by signal generators 13 to 17 can be adjusted in frequency, pulse width, amplitude and repetition rate, or with respect to any other variable. Coupled to generators 13 to 17 and activated by a signal applied thereto at terminal 20A is a mechanical, electronic, or optical, etc. (e.g., nanomaterial) selector switch 20. The output signal from the signal generator selected by a switch 20 is applied through a line 21 to output stage 11. In practice, the line is preferably a bus system.

The ES signal stage 10 is preferably miniaturized and may take the form of a hybrid device or a single ASIC chip (Application Specific Integrated Circuit). Output stage 11 includes a mechanical, electronic, or optical selector switch 22 which applies the ES signal from stage 10 either to a low voltage processor 23, a high voltage processor 24, a current processor 25, or a power processor 26 to put the ES signal in a form appropriate to the intended application for electrical stimulation. In a preferred version, all signals can be accessed simultaneously by one or more output stages through a system bus. In practice, a combination of one or more signal generators in the ES signal stage with one or more of the output stages can be miniaturized.

The output of the processor 23, 24, 25 or 26 chosen by selector switch 22 is fed to a modulator 27 coupled to an amplitude control unit 28 which modifies the amplitude of the signal applied thereto. (There are also other ways to control the amplitude.) The output of amplitude-control unit 28 is applied to a polarity control unit 29 in which the electrical stimulation signal is given a positive or negative polarity or is converted to an AC signal, depending on the intended application for the electrical stimulation signal.

Each output stage can be configured with either multiple output terminals 30 or with a single output. The multiple outputs make it possible to run several parallel experiments or processes concurrently.

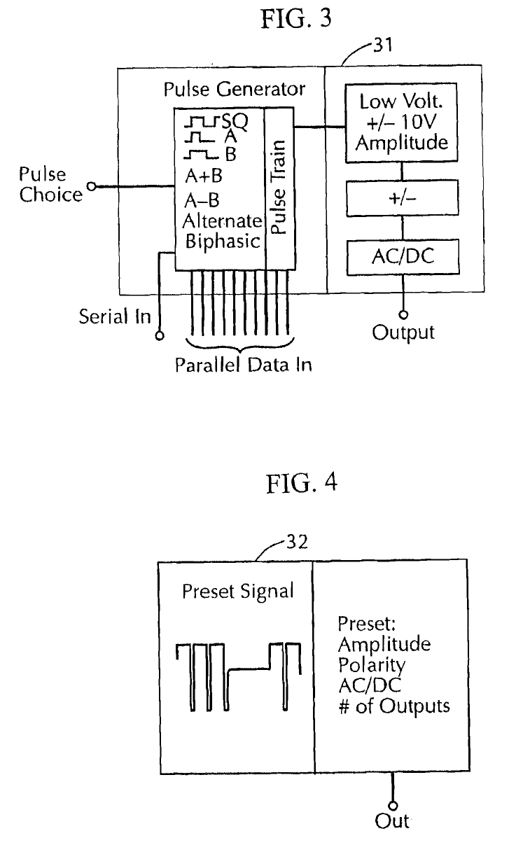

As previously mentioned, the ES system can be miniaturized to form a single ES component comprising signal generators and miniaturized output circuitry packaged together. A functional sketch of one such ES component 31 is shown in FIG. 3, and an example of a customized module 32 with a preset waveform and preset electrical parameters is shown in FIG. 4.

A preferred version of the ES component includes a sophisticated digital pulse generator on a chip and an analog circuitry to define complex pulse patterns, with amplitudes appropriate for a given application. The output can be fed into any number of desirable output stages, which can be integrated into the same component or be independent proprietary devices, e.g., voltage controlled or current controlled output stages with various voltage/current amplitudes, high frequency output stage with various bandwidths depending on a specific application, various power output stages, etc. Waveforms other than pulse patterns, as well as modulated signals can be part of such a “system on a chip.”

The design of a digital pulse generator (which can be implemented, e.g., as an ASIC) consists of several blocks, which can be either used together to create a sophisticated pulse generator for biomedical applications, or can be used in any number of other applications requiring a pulse signal. Each of these blocks or functional modules can provide an independent waveform or pulse (A pulse; B pulse; square wave; time delay; etc). A basic one output version of the signal generator delivers two independent pulses A and B with digitally adjustable pulse widths, the same pulse repetition rate, and with an adjustable delay between them or for each of them. It also delivers a square wave and timing for alternate and biphasic pulses and two pulse trains. In a two or more output version, individual pulses can have independently set repetition rates.

Several of these independent signal generators can be combined into a multi-output device. All timing parameters of the pulses preferably are fully programmable by a user via hardware or via software. For example, one can adjust timing using thumbwheels or switches connected via parallel inputs of the ES component, or by using software and a serial, parallel, or custom interface as an input (or a combination of analog and digital inputs can be used). The ES component can include both a parallel and a serial interface so that the user can define the optimal means for each application.

The analog output amplitudes of the ES component or ES system can be adjusted for each pulse separately (via hardware or software, as above). At the same time, a specific DC level can be added; i.e., signal can be shifted up or down from zero line. The alternate and biphasic pulses are designed so that only one adjustment for both positive and negative pulse width and amplitude is required, which results in guaranteed symmetrical signals.

Electric stimulator components, multi-functional systems, and methods for generating output signals are described, for example, in above-mentioned U.S. Pat. No. 6,684,106, which is hereby incorporated by reference herein in its entirety.

Referring back to FIG. 1, delivery system 100 may also include a feedback mechanism. One or more sensors may be connected to delivery system 100. The sensors may be used to measure and/or detect various conditions prevailing in the tissue or any suitable medium. Sensor interface 106 may include any component necessary to monitor and analyze data received by the sensors. For example, as shown in FIG. 1, sensor interface 106 includes signal conditioning circuitry and data acquisition circuitry. In response to receiving a signal from a sensor, the feedback mechanisms may use the information relating to, for example, the pH level, the pO₂ level, the volume of drugs distributed, the drug dosage, timing parameters for drug delivery (e.g., time-released, delayed release, etc.), or any other suitable parameter and direct the stimulator, the controller, or any other suitable component of delivery system 100 to perform an operation. For example, the stimulator may use at least a portion of the information to adjust its signal accordingly to maintain an optimal level of measured entity (e.g., pH, pO₂, ion concentration, biochemical marker, pressure, temperature, etc.) and to optimize the treatment outcome. In particular, the sensor capability and a feedback loop allow a user to measure or to measure automatically the effects of electrical stimulation on the tissue under treatment and also to adjust the level, the type of the electrical stimuli, and/or any other suitable parameter to optimize the treatment outcome. Using the drug delivery system, external or implantable devices for drug delivery may also be controlled.

It should be noted that the delivery system of the present invention is also capable of analyzing and/or controlling linear and non-linear processes, such as flow, pressure, temperature, and pH. For example, the delivery system of the present invention may include an adaptive controller or a model free adaptive controller. Because proportional-integral-derivative (PID) controllers may not be able to handle large dynamic changes (e.g., the logarithmic changes of pH), the model free adaptive controller may be used.

In some embodiments, the electrical stimulator component of the delivery system may be used to control the delivery of drugs, fluids, or other substances. Using the electrical stimulator component, several, simple or complex rectangular pulses and other signals are available, for the output stages, through a selector or preferably on a bus. These signals are synchronized and may be accessed by an output stage either by selecting only one signal or by accessing several signals at the same time. In the latter case, the signals may be combined in the output stage to a more complex pattern. In either case, they may be accessed by a single selected output stage or simultaneously by several, either identical or different, output stages.

For example, the electrical stimulator component may include a signal generator that generates multiple signals, where each signal has a controllable waveform based on one or more electrical parameters (e.g., amplitude, frequency, shape, timing parameters, phase, pulse duration, and pulse repetition rate). The electrical stimulator component may also include a selection mechanism to select as output signals one or more the signals generated by the signal generator. These output signals are placed on one or more output terminals. For example, multiple signals (e.g., a combined signal) may be placed on a single output terminal. In another example, identical signals may be generated and placed on different output terminals. In yet another example, different signals may be generated using the signal generator and placed on different output terminals. The output signals control the release of the substance from the reservoirs connected to the delivery system.

It should be noted that the electrical stimulator is configurable such that the delivery of substances in reservoirs connected to the delivery system may be synchronized and/or preset (e.g., daily doses, intermittent, maintenance of treatment of a daily basis, etc.), fluid flow from reservoirs may be controlled such that precision mixture of various substances (e.g., fluids or drugs) may be attained, substances may be time delayed or time released, substances may be volumetrically controlled, substances may be diluted to provide an appropriate dosage, etc. For example, the electrical stimulator may be configured to generate two identical signals on different output ports that control reservoirs containing different substances. Upon receiving the identical signals, the delivery system may synchronously dispense two different substances at the same time.

Using the electrical stimulator component, the delivery system may be used for a number of applications when combined with different output stages. The delivery system may be used as a controller in an automatic external or implantable drug delivery device. The delivery system may be preset with predefined signal patterns for specific application or for double blind studies, thereby eliminating the need to continuously develop new experimental systems at a larger cost.

The electrical stimulator component may be integrated in or connected to an external or implantable device that holds a given amount of drugs, fluids, or any other suitable substance (e.g., in one or more reservoirs). For example, electrodes that are connected to the electrical stimulator component may be implanted in a tumor or placed in its immediate vicinity and current is caused to flow therethrough between the electrodes in the course of which a cytotoxic agent is synthesized in situ by a reaction between the material of the electrode and a substance delivered to the tumor.

In practice those or other reactions may take place between the electrode and the reagent introduced into the tumor, with a hollow electrode or a catheter used for delivery, or between two or more substances either naturally occurring in the organism or introduced into the tumor, with the electrode having an electrocatalytic effect. The material of the electrode controls the course and/or the speed of reactions whose products are cytotoxic.

One way of carrying out a method is by potential-controlled reaction at the electrodes implanted in the tumor. Another way in accordance with the invention is by using hollow electrodes or a catheter for adding an appropriate substance to ensure the proper chemical environment for the formation of the cytotoxic agent in situ. Still another way is by continuously or intermittently releasing in situ from an implanted reservoir (one embodiment is a hydrogel capsule, as described below) appropriate substances to ensure the proper chemical environment for the formation of the cytotoxic agent in situ. Such time released substances, activated electrically when needed, can be applied over extended periods of time including a period after the main treatment has been completed, to prevent a reoccurrence of cancer. These can also be used to activate the immune system response.

The same method can be used to produce in situ non-cytotoxic agents, which can later be converted to the cytotoxic agents by isomerization. Isomerization is a process whereby a compound is changed into one of its isomers; i.e., one of two or more chemical substances having the same elementary percentage composition and molecular weight but differing in structure, and therefore in properties. An example thereof is transplatin and cisplatin. While cisplatin is highly cytotoxic, transplatin is considered not to be.

Because the synthesis process is electrically-assisted and because the electrical stimulator component is configurable (e.g., amplitude, frequency, shape, timing parameters, phase, pulse duration, and pulse repetition rate), the electrical stimulator component of the delivery system may be used to control the amount of crytotoxic agent, which is synthesized.

Additional systems and methods for in situ electrochemical treatment of a malignant tumor, which destroys the tumor with minimal damage to regions surrounding the tumor, are described further in commonly-owned, commonly-assigned U.S. Pat. No. 6,708,066, which is hereby incorporated by reference herein in its entirety.

In some embodiments, the delivery system of the present invention may be used to automatically control the release of various substances from a device, such as an implantable device. For example, an implantable reservoir carrying one or more drugs, fluids, or other suitable substances may be automatically controlled by means of electrical modulation of the material properties of the walls of such a reservoir, for example a hydrogel capsule, or by controlling a micro pump, e.g., an insulin pump with an appropriate time sequence of an electric signal.

In some embodiments, the sensors used in the delivery system may be hydrogel biosensors. Hydrogel biosensors (such as those developed by M-Biotech, Inc. of Salt Lake City, Utah) may be used for the continuous monitoring of various substances, such as, for example, salt, pH, temperature, alcohol, amino acids, flavors, penicillin etc. Each hydrogel biosensor generally consists of two parts: a hydrogel and a miniature pressure transducer. In case of a glucose biosensor, the hydrogel either swells or contracts as a function of glucose concentration changes in the body fluid. A pressure transducer is used to measure the changes in the swelling of the hydrogel. A resulting voltage signal (in mV) is produced on the output of the transducer.

In some embodiments, the sensors used in the delivery system may be self-assembling hydrogels or any other suitable self-assembling substance. These self-assembling hydrogels may include two materials (e.g., fluids) that remain separate until, for example, a given pH level is reached. Upon reaching that pH level, the two materials self-assemble into a hydrogel. This pH-dependent hydrogel may also disassemble at a specific pH level. These self-assembling hydrogels may be used as configurable membranes or reservoirs for drug delivery. For example, upon connecting the delivery system to control a self-assembling hydrogel, the delivery system may be used to control the pH level of the hydrogel such that at a given pH (or other suitable parameter), the hydrogel assembles preventing and/or significantly reducing the amount of drugs or any suitable substance released to the area where the hydrogel is located. Similarly, a given pH may be attained such that the hydrogel dissembles at predefined pH and allows the drugs or any suitable substance to be administered. The delivery system having the electrical stimulator may be used to control the permeability of a given area or delivery port.

For example, a number of self-assembling hydrogel reservoirs may be fabricated. Upon connecting each output port of the electrical stimulator component or any other suitable component to each self-assembling hydrogel reservoir, the delivery system is capable of controlling the release of each of the substances contained in each reservoir (e.g., each reservoir contains 1 mL of a given drug and when 4 mL of the drug are needed, four output signals are transmitted through the output ports to effect the release of four reservoirs).

In another suitable example, the reservoir may be made of any suitable material, where the reservoir has an opening (having a given dimension) made of the self-assembling hydrogel. For example, an electrode may be weaved through the self-assembling hydrogel such that the assembling and disassembling of the hydrogel may be electrically controlled. By controlling the hydrogel opening, the reservoir may release a given amount of drugs for a predefined amount of time.

In the example of treating tissues, such as a malignant tumor, a hydrogel capsule may be used to continuously or intermittently release in situ from an implanted reservoir appropriate substances, such as drugs, to ensure the proper chemical environment for the formation of the cytotoxic agent in situ. Such time released substances, activated electrically when needed, may be applied over extended periods of time including a period after the main treatment has been completed to prevent a reoccurrence of cancer.

Accordingly, in some embodiments, the delivery system may be used to automatically adjust an electrical stimulation signal to optimize the treatment outcome in response to receiving a specific parameter (e.g., biomarker, time, volume, dosage, pH, etc.) measured over time.

It should be noted that the delivery system according to the present invention may include a general purpose computer, a specially programmed special purpose computer, or a microprocessor embedded in the system. A user may interact with the system via e.g., a personal computer or over PDA, e.g., the Internet, an Intranet, or embedded web interface, etc. Either of these may be implemented as a distributed computer system rather than a single computer. Similarly, the communications link may be a wireless link, a dedicated link, a modem over a POTS line, the Internet and/or any other method of communicating between computers and/or users. Moreover, the processing could be controlled by a software program on one or more computer systems or processors (e.g., without the electrical stimulator component, etc.), or could even be partially or wholly implemented in hardware.

It should be noted that the resulting delivery system may be implemented as an electronic component that is optimized for size, cost and performance and constitutes a main building block for various delivery devices. Alternatively, the delivery system may also be available as an electronic component with a sensor input and a feedback loop.

It is to be understood that the invention is not limited in its application to the details of construction and to the arrangements of the components and systems set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Although the present invention has been described and illustrated in the foregoing exemplary embodiments, it is understood that the present disclosure has been made only by way of example, and that numerous changes in the details of implementation of the invention may be made without departing from the spirit and scope of the invention, which is limited only by the claims which follow.

What is claimed is:

1. A system for delivering a substance in a medium, the system comprising:
a delivery component that includes at least one reservoir, wherein the at least one reservoir contains the substance;
an electronic controller that is connected to the delivery component and that controls the release of the substance from the at least one reservoir into the medium; and
a sensing mechanism that monitors at least one parameter in the medium and that provides a signal to a feedback mechanism, wherein the feedback mechanism sends an electrical control signal to the controller to effect the release of the substance from the at least one reservoir into the medium.

2. The system of claim 1, wherein the electronic controller further comprises:
at least one signal generator for generating a plurality of signals, wherein each signal has a controllable waveform based on at least one electrical parameter, and wherein the at least one electrical parameter includes any of: amplitude, frequency, shape, timing parameters, phase, pulse duration, and pulse repetition rate;
a selection mechanism for selecting as output signals one or more of the plurality of generated signals; and
at least one output terminal, wherein the electronic controller is configured to place at least one of the output signals on the at least one output terminal and wherein the output signals control the release of the substance from the at least one reservoir.

3. The system of claim 1, wherein the sensing mechanism comprises any of sensing electrodes, pickup coils, temperature sensitive devices, magnetic probes, biosensors, and biomarkers.

4. The system of claim 1, wherein the electronic controller is further configured to compare the monitored parameter to a desired value for the at least one parameter.

5. The system of claim 4, wherein the desired value is one of an upper limit and a lower limit for the monitored parameter.

6. The system of claim 1, wherein the system is one of an electronic component, an integrated circuit, a multi-chip module, a hybrid circuit, a system-on-chip (SoC), a system-in-package (SiP), and a lab-on-a-chip.

7. The system of claim 1, wherein the controller is further configured to send an electrical control signal that effects the release of a given volume of the substance from the at least one reservoir.

8. The system of claim 1, wherein the controller is further configured to send an electrical control signal that effects the release of the substance from the at least one reservoir for a given time sequence or time pattern.

9. The system of claim 1, wherein, in response to the sensing mechanism detecting that the concentration of the substance in the at least one reservoir is greater than a desired value, the electronic controller is configured to send an electrical control signal that effects the release of another substance into the at least one reservoir that dilutes the substance.

10. The system of claim 1, wherein, in response to the sensing mechanism detecting that the dosage of the substance in the at least one reservoir is greater than a desired value, the electronic controller is configured to send an electrical control signal that effects the amount of the substance that is released from the at least one reservoir.

11. The system of claim 1, wherein the at least one parameter comprises any of pH, pO₂, volume, pressure, temperature, ion, dosage, time, impedance, and a biomarker.

12. The system of claim 1, wherein the system is a drug delivery system.

13. A system for delivering substances in a medium, the system comprising:
a delivery component that includes a plurality of reservoirs, wherein each of the plurality of reservoirs contains a substance;
a controller that is connected to the delivery component, wherein the controller comprises:
at least one signal generator for generating a plurality of signals, wherein each signal has a controllable waveform based on at least one electrical parameter, and wherein the at least one electrical parameter includes any of: amplitude, frequency, shape, timing parameters, phase, pulse duration, and pulse repetition rate;
a selection mechanism for selecting as output signals one or more of the plurality of generated signals; and
a plurality of output terminals, wherein the controller is configured to place at least one of the output signals on each of the output terminals and wherein the output signals control the release of the substance from the plurality of reservoirs;
a sensing mechanism that monitors at least one parameter in the medium; and
a feedback mechanism that directs the controller to effect the release of at least one of the substances from the plurality of reservoirs into the medium based at least in part on the monitored parameter.

14. The system of claim 13, wherein the controllable waveform comprises pulses.

15. The system of claim 13, wherein the at least one parameter comprises any of pH, pO₂, volume, pressure, temperature, ion, dosage, time, impedance, and a biomarker.

16. The system of claim 13, wherein the controller further comprises a combining mechanism for combining two or more generated signals to provide the output signal.

17. The system of claim 13, wherein the sensing mechanism comprises any of sensing electrodes, pickup coils, temperature sensitive devices, magnetic probes, biosensors, and biomarkers.

18. The system of claim 13, wherein the system is one of an electronic component, an integrated circuit, a multi-chip module, a hybrid circuit, a system-on-chip (SoC), a system-in-package (SiP), and a lab-on-a-chip.

19. The system of claim 13, wherein the plurality of signals are identical and wherein the controller is further configured to place each of the plurality of identical signals on each of the plurality of output terminals.

20. The system of claim 19, wherein the controller is capable of synchronizing the plurality of identical signals.

21. The system of claim 13, wherein the controller is arranged to independently place each of the signals on the output terminals such that each signal on each of the output terminals is independent from signals on each of the other output terminals.

22. The system of claim 21, wherein the controller is capable of synchronizing the plurality of independent signals.

23. The system of claim 13, wherein the controller is arranged to independently place each of the signals on the output terminals such that at least one of the signals on an output terminal is independent from another signal on another output terminal.

24. The system of claim 23, wherein the controller is capable of synchronizing the at least one signal and the another signal.

25. The system of claim 13, wherein the substance in one of the reservoirs is identical to the substance in each of the other reservoirs.

26. The system of claim 13, wherein the substance in one of the reservoirs is different from the substance in each of the other reservoirs.

27. The system of claim 13, wherein the substance in one of the reservoirs is different from the substance in another reservoir.

28. The system of claim 13, wherein the controller is further configured to send an electrical control signal that effects the release of a given volume of the substance from one of the plurality of reservoirs.

29. The system of claim 13, wherein the controller is further configured to:
send a first electrical control signal that effects the release of a first volume of a first substance from one of the reservoirs; and
send a second electrical control signal that effects the releases of a second volume of a second substance from another reservoir, wherein the first electrical signal and the second electrical signal are synchronized and wherein the first substance and the second substance are mixed.

30. The system of claim 29 wherein said controller is further configured to:
send a sequence of control signals for at least one of delivering and mixing a plurality of substances from said reservoirs.

31. The system of claim 13 wherein said controller is further configured to:
send a first electrical control signal that effects the release of a first volume of a first substance from one of the reservoirs;
send a second electrical control signal that effects the releases of a second volume of a second substance from another reservoir, wherein the first electrical signal and the second electrical signal are synchronized; and
send said control signals that effect the release of said substances in parallel or in sequence.

32. The system of claim 13, wherein the controller is further configured to send an electrical control signal that effects the release of the substance from one of the plurality of reservoirs for a given amount of time.

33. The system of claim 13, wherein, in response to the sensing mechanism detecting that the concentration of the substance in at least one reservoir of the plurality of reservoirs is greater than a desired value, the controller is further configured to send an electrical control signal that effects the release of another substance into the reservoir that dilutes the substance for a given time sequence.

34. The system of claim 13, wherein, in response to the sensing mechanism detecting that the dosage of the substance in at least one reservoir of the plurality of reservoirs is greater than a desired value, the controller is further configured to send an electrical control signal that changes the amount of the substance that is released from the at least one reservoir.

35. A method for delivering a substance in a medium, the method comprising:
receiving a signal from at least one sensor, wherein the at least one sensor monitors at least one parameter in the medium; and
in response to receiving the signal from the at least one sensor, transmitting an electrical control signal derived at least in part from the at least one sensor to a controller to effect the release of the substance from at least one reservoir into the medium.

36. A method for delivering substances in a medium, the method comprising:
providing a plurality of reservoirs;
generating a plurality of signals, wherein each signal has a controllable waveform based on at least one electrical parameter, and wherein the at least one electrical parameter includes any of: amplitude, frequency, shape, timing parameters, phase, pulse duration, and pulse repetition rate;
selecting one or more of the plurality of generated signals for use as output signals;
providing multiple output terminals, wherein at least one of the multiple output signals is placed on each output terminals and wherein the output signals control the release of substances from the plurality of reservoirs;
monitoring at least one parameter in the medium;
directing at least one controller that is connected to the plurality of reservoirs to effect the release of at least one of the substances from the plurality of reservoirs into the medium based at least in part on the monitored parameter.

37. The method of claim 35 or 36, wherein the at least one parameter is a biomarker.

38. A method for delivering a substance to ectoderm-derived tissue, the method comprising:
receiving a signal from at least one sensor, wherein the at least one sensor monitors at least one parameter in the tissue; and
in response to receiving the signal from the at least one sensor, transmitting an electrical control signal derived at least in part from the at least one sensor to a controller to effect the release of the substance from at least one reservoir to the tissue.

39. The method of claim 38 further comprising:
(a) generating a plurality of signals, wherein each signal has a predetermined waveform and at least one of the signals comprises pulses;
(b) adjusting one or more electrical parameters of at least one signal, wherein the parameters include any of: amplitude, frequency, shape, timing parameters, phase, pulse duration, and pulse repetition rate; and
(c) selecting as the control signal one or more of the generated and adjusted signals, wherein the selection is based upon an intended application.

40. The method of claim 38, further comprising
(a) sensing, using one or more sensors, at least one measurable condition in the subject and providing a sensor output; and
(b) adjusting one or more electrical parameters of at least one signal responsive to the sensor output, wherein the parameters include any of: amplitude, frequency, shape, timing parameters, phase, pulse duration, and pulse repetition rate.

41. The method of claim 38 , wherein the ectoderm-derived tissue is nervous tissue, comprising at least one of brain, spinal cord, optic, and peripheral nerves.

42. The method of claim 39, wherein the intended stimulation application is regeneration or repair.

43. The method of claim 39, wherein the intended stimulation application is pain relief.

44. The method of claim 39, wherein the intended stimulation application is recovery or maintenance of at least one of upper and lower extremity function, bladder, bowel, and erectile function, and respiratory function.

45. A method for delivering a substance to mesoderm-derived tissue, the method comprising:
receiving a signal from at least one sensor, wherein the at least one sensor monitors at least one parameter in the tissue; and
in response to receiving the signal from the at least one sensor, transmitting an electrical control signal derived at least in part from the at least one sensor to a controller to effect the release of the substance from at least one reservoir to the tissue.

46. The method of claim 45 further comprising:
(a) generating a plurality of signals, wherein each signal has a predetermined waveform and at least one of the signals comprises pulses;
(b) adjusting one or more electrical parameters of at least one signal, wherein the parameters include any of: amplitude, frequency, shape, timing parameters, phase, pulse duration, and pulse repetition rate; and
(c) selecting as the control signal one or more of the generated and adjusted signals, wherein the selection is based upon an intended stimulation application.

47. The method of claim 45, further comprising
(a) sensing, using one or more sensors, at least one measurable condition in the subject and providing a sensor output; and
(b) adjusting one or more electrical parameters of at least one signal responsive to the sensor output, wherein the parameters include any of: amplitude, frequency, shape, timing parameters, phase, pulse duration, and pulse repetition rate.

48. The method of claim 45, wherein the mesoderm-derived tissue is at least one of bone, circulatory system, muscle, and urinary system tissues.

49. The method of claim 46, wherein the intended stimulation application is regeneration or repair.

50. The method of claim 40 or 47, wherein the at least one measurable condition is a biomarker.

51. The method of claim 40 or 47, wherein the sensor output further comprises a comparison of the sensed measurable condition and a desired value or range of values for the measurable condition.

52. The method of claim 36, further comprising recording the monitored parameter and at least one of the amount, substance, and time of the effected release.

53. The method of claim 40, further comprising recording the sensed measurable condition and the adjusted electrical parameters.

54. The method of claim 47, further comprising recording the sensed measurable condition and the adjusted electrical parameters.

55. The method of any of claims 52-54, further comprising repeating the method and comparing the new recorded information to the previously recorded information, for at least one of diagnosis or treatment.
